(19) United States
(12) Patent Application Publication
(10) Pub. No.: US 2008/0167581 A1
(43) Pub. Date: Jul. 10, 2008

(54) DETERMINING PARAMETERS ASSOCIATED WITH A FEMALE PELVIS AND CERVIX

(76) Inventor: Yoav Paltieli, Haifa (IL)

Correspondence Address: DEKEL PATENT LTD., DAVID KLEIN, BEIT HAROF'IM, 18 MENUHA VENAHALA STREET, ROOM 27, REHOVOT, 76209, omitted

(21) Appl. No.: 11/621,625

(22) Filed: Jan. 10, 2007

(51) Int. Cl. A61B 5/103

(52) U.S. Cl. 600/588

(57) ABSTRACT

A method for calculating fetal parameters with reference to a female pelvis, the method including previously obtaining measurement of a pelvis of a woman, re-locating the pelvis by sensing a point on the pelvis and finding a spatial orientation of the point with the previously measured pelvis and one other reference point, and calculating a fetal parameter of a fetus in the woman with respect to the pelvis.

### FIELD OF THE INVENTION

The present invention relates to a method and apparatus for monitoring the progress of labor during childbirth, and particularly to methods and apparatus for determining the dimensions and the spatial position of the female pelvis, and for determining cervical information associated with a pregnant woman.

### BACKGROUND OF THE INVENTION

Normal labor is generally divided into three stages: the first stage begins with the onset of labor and ends when dilatation of the cervix is complete; the second stage begins at that point and ends with the complete birth of the baby; and this is followed by the third stage which ends with the delivery of the placenta. During labor it is common to use either an external ultrasonic system for recording the baby's heart rate, and an external system for detecting the mother's uterine contractions, or an electronic system to sense the baby's heart pulses by an electrode attached to the baby's head and the mother's contractions by a pressure catheter applied to the mother inside the uterus.

However, a number of other physiological conditions of the mother and baby during labor can also be monitored in order to determine the progress of labor. These additional conditions include: (1) effacement (the thinning out of the cervix that occurs before and during the first stage of labor); (2) cervical dilatation (the increase in size of the cervical opening); (3) position of the cervix (the relation of the cervix to the vaginal axis, normally the fetal head); (4) station (the level of a predetermined point of the fetal presenting part with reference to the mother's pelvis), (5) position of the head which describes the relationship of the head to the pelvis and (6) and presentation which describes the part of the fetus (such as brow, face or breech) at the cervical opening.

The more common determination of station is the distance between the tip of the fetal head and the ischial spines which can be palpable by the physician; but a more accurate determination of station is the distance between the bi-parietal diameter (BPD) of the fetal head and the mother's pelvic inlet.

The foregoing conditions are generally determined by a physical examination, e.g., by the insertion of a finger through the mother's vagina. However, the accuracy of such a “finger” examination is very subjective and depends to a great extent on the experience, judgment, and even finger size, of the physician. Other drawbacks in such a physical examination are that it can be done only at spaced intervals, it generally produces discomfort to the mother, and it involves a number of risks including contamination, infection, dislodgment of a fetal monitor, injury to the baby, etc. Failure to interpret the precise stage of the labor progress from the physical examination can result in injury or even death of the baby or of the mother.

U.S. Pat. No. 6,200,279 to Paltieli, incorporated herein by reference in its entirety, describes improved methods and apparatus for monitoring the progress of labor. In one embodiment, the progress of labor is monitored by attaching a position sensor to a predetermined point on the mother's pelvic bones, monitoring the location of the position sensor in three-dimensional space relative to a reference, and monitoring the location of the fetal presenting part with respect to the predetermined point on the mother's pelvic bones. The location of the fetal presenting part may be indicated by a similar position sensor, or by imaging. Other conditions, such as effacement, cervical dilatation, and cervical position may also be monitored in a similar manner.

In U.S. Pat. No. 6,669,653, a continuation-in-part application of U.S. Pat. No. 6,200,279, further embodiments are described. According to one aspect of U.S. Pat. No. 6,669,653, incorporated herein by reference in its entirety, monitoring the location of the fetal presenting part with respect to the predetermined point on the mother's pelvic bones provides an indication of the progress of labor; and the cervical dilation may be measured by attaching sensors to the cervix.

In another embodiment of U.S. Pat. No. 6,669,653, there is provided a method of non-continuous monitoring of the progress of labor in a mother during childbirth, comprising: using a probe or finger-mounted sensor to measure the fetal presenting part relative to a predetermined point on the mother's pelvic bone, and to measure the cervical dilation by touching the cervix in, for example, two points.

In another embodiment, the locations of the fetal presenting part and of the opposite sides of the end of the mother's uterine cervix may be monitored by position sensors attached to these respective elements. In a second described embodiment, the latter are monitored non-continuously using a hand held probe or finger-mounted sensor. In a third described embodiment, the latter are monitored by operating an ultrasonic transducer to image the mother's cervix and pelvic bones, and the fetal head, on a screen, and by using a position sensor on the ultrasonic transducer, and a marker for marking the screen, to locate the positions of these elements. A fourth embodiment is described utilizing at least two sensors, one of which is attached to a bony position on the pelvis to serve as the reference point, and the others may first be used to map the pelvis from outside of the body and to map the BPD plane by attaching it to the ultrasonic probe, to map the ischial spines and ischial tuberosities from the inside and then to be attached to the cervix and fetal presenting part.

In a further embodiment of U.S. Pat. No. 6,669,653, position sensors may also be attached to, or position coordinates may be obtained of, the anterior superior iliac spine, the pubic symphysis, the scrum at 1-3 levels, the ischial spines and the ischial tuberosity, and such positions may be used for mapping the pelvic inlet, outlet and midpelvis. Such mapping or pelvimetry may be helpful in determining whether the head of the baby is of suitable size for passage through the birth canal.

According to further features in U.S. Pat. No. 6,669,653, the cervical dilatation of the mother's cervix is continuously indicated by monitoring the positions of the position sensors applied to the opposite sides of the end of the cervix, and continuously displaying the spatial distance between them. The position of the fetal presenting part (e.g., fetal head) is also continuously indicated by monitoring and displaying their respective locations. In another embodiment, the cervical dilatation of the mother's cervix and the position of the fetal presenting part or the BPD are monitored on a non-continuous basis by touching a probe or finger-mounted sensor to each side of the cervix and a pre-determined point or points on or connected to the fetal head.

According to further features in U.S. Pat. No. 6,669,653, the above conditions are computed and displayed in the form of units of distance (e.g., cm), and/or in the form of a graph (e.g., partogram), showing the interrelation of the cervical dilatation and the descent of the fetal presenting part. Furthermore, such a display may include an image of the fetus within the birth canal and the relation and orientation over time of the head to the pelvic inlet, outlet and midpelvis. Other methods to display such information may be used.

The methods and apparatus of U.S. Pat. No. 6,669,653 permit monitoring the progress of labor in a manner which is either continuous or intermittent, which is less dependent for accuracy on the experience, judgment or finger size of the attendant in the conventional “finger examination”, which subjects the mother to less discomfort, and which involves less risk of contamination, infection, dislodgment of a fetal monitor, or injury to or death of the baby or mother due to a wrong assessment of the fetal position or of labor progress. Moreover, this technique enables more precise monitoring of the critical condition, namely the changes in the spatial distance of the BPD of the baby's head with respect to the pelvic inlet.

### SUMMARY OF THE INVENTION

The present invention seeks to provide methods and apparatus for determining the dimensions and the spatial position of the female pelvis and for determining cervical information associated with a pregnant woman, as is described more in detail hereinbelow.

Imaging modalities and position sensors may be used to map the female pelvis, as part of a process to define fetal head station and position. The present invention describes a number of different methods of determining the dimensions and spatial position of the female pelvis, and specifically the pelvic inlet. The dimensional and positional information may be utilized as a reference to define fetal head station and position by methods described hereinbelow.

In one embodiment of the present invention, methods and apparatus are provided for the use of imaging modalities and position sensors to measure the length of the cervix during early labor. In another embodiment of the present invention, a new method is provided for determining the cervical dilatation in the active phase of labor utilizing ultrasound in the infrapubic approach, as is described more in detail herein below. In another embodiment of the present invention, a new method is provided for determining the cervical dilatation and length during labor utilizing ultrasonic markers, as is described more in detail herein below.

There is thus provided in accordance with an embodiment of the present invention a method for calculating fetal parameters with reference to a female pelvis, the method including previously obtaining measurement of a pelvis of a woman, re-locating the pelvis by sensing a point on the pelvis and finding a spatial orientation of the point with the previously measured pelvis and one other reference point, and calculating a fetal parameter of a fetus in the woman with respect to the pelvis. The fetal parameter may be, for example, the station or position of a head of the fetus.

Re-locating the pelvis may include attaching a reference sensor to a known point on the pelvis, and the other reference point may include at least one additional known point of the pelvis. The at least one additional known point of the pelvis may be acquired by touching that point with a position sensor or by marking the point with an ultrasonic marker.

The method may include using additional positional information about the pelvis to determine a spatial position of the pelvis and to calculate the fetal parameter. The additional positional information may be acquired by obtaining a vertical plane of the pelvis, or by a direction of finger insertion during a digital vaginal examination.

Re-locating the pelvis may include marking at least two known points of the pelvis, and the method may include performing an examination of the fetus in the woman without maternal movement between the marking and completion of the examination. The method may further include making a statistical average angle of a line between two pelvic features and orienting pelvic planes along said angle and a line created by marking two points on the pubic bone.

There is also provided in accordance with an embodiment of the present invention a method for determining fetal head station in a birth canal associated with a female pelvis of a woman pregnant with a fetus, the method including making an ultrasonic image that includes the birth canal and the pelvis of the woman, identifying a tip of a skull of the fetus, marking known landmarks of the pelvis associated with the birth canal, which are seen on the ultrasonic image, and calculating head station by correlating the known landmarks with the tip of the skull (e.g., by marking an intersection of the skull and a line representing the birth canal).

The head station may be calculated as a distance to a pelvic feature, such as pelvic inlet, midpelvis, pelvic outlet, ischial spines, a plane perpendicular to an inlet plane of the pelvis, or as a distance on a line of the birth canal. Marking the intersection of the skull and the line representing the birth canal may be done at a constant radius from a symphysis pubis of the pelvis. The method may further include processing the ultrasonic image to automatically identify known landmarks of the pelvis and the skull.

There is also provided in accordance with an embodiment of the present invention a method for determining cervical information associated with a woman pregnant with a fetus, the method including identifying a cervix on an ultrasonic image, marking boundaries of the cervix, calculating cervical length and dilatation based on the identified cervix and its boundaries, and creating and displaying a three-dimensional reconstruction of the cervix.

The method may further include identifying a leading edge of a skull of the fetus. The method may further include searching the ultrasonic image for a first set of pixels that move over a skull of the fetus during a uterine contraction (in a downward direction at the beginning of contraction and upward at the end of the contraction), attributing the first set of pixels as relating to the skull descending from a force of the uterine contraction, and attributing a second set of pixels in the ultrasonic image, which move generally perpendicularly with respect to the first set of pixels, as related to the cervix dilating during the uterine contraction.

There is also provided in accordance with an embodiment of the present invention apparatus for determining dimensions and spatial position of a female pelvis, the apparatus including a reference sensor attachable to a known point on a female pelvis of a pregnant woman, a position sensor for acquiring a position of at least one additional known point of the pelvis, and ultrasonic markers of at least two known points of the pelvis.

There is also provided in accordance with an embodiment of the present invention apparatus for determining fetal head station in a birth canal associated with a female pelvis of a woman pregnant with a fetus, the apparatus including an ultrasonic image that includes the birth canal and the pelvis of the woman, an ultrasonic identifier of a tip of a skull of the fetus, ultrasonic markers of known landmarks of the pelvis associated with the birth canal, which are seen on the ultrasonic image, and a processor for calculating head station that correlates the known landmarks with the tip of the skull.

The processor may be adapted to process the ultrasonic image to automatically identify known landmarks of the pelvis and the skull.

There is also provided in accordance with an embodiment of the present invention apparatus for determining cervical information associated with a woman pregnant with a fetus, the apparatus including an ultrasonic image that includes a cervix identified thereon, a processor for calculating cervical length and dilatation based on the identified cervix and its boundaries, the processor being operative to create a three-dimensional reconstruction of the cervix. A display may be provided for displaying the three-dimensional reconstruction of the cervix.

### BRIEF DESCRIPTION OF THE DRAWINGS

The present invention will be understood and appreciated more fully from the following detailed description taken in conjunction with the drawings in which:

FIG. 1 is a simplified illustration of a method for measuring and re-locating a female pelvis, in accordance with an embodiment of the present invention;

FIG. 2 is a simplified illustration of a method for determining the fetal head station in the birth canal, in accordance with an embodiment of the present invention;

FIGS. 3-5 are simplified illustrations of different stages of the cervix during labor, wherein FIG. 3 illustrates the cervix as an elongated cylindrical structure in the early stages of labor, as seen by trans-perineal ultrasound, appearing in an ultrasonic image as two echogenic layers separated by a white line, FIG. 4 illustrates the cervix open on its proximal end (“funneling”), and FIG. 5 illustrates the active phases of labor, wherein the cervix is effaced and is a flat structure covering the skull with a round opening at the center; and

FIG. 6 is a simplified illustration of a method for ultrasonic identification of the cervix, in accordance with an embodiment of the present invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be appreciated by one skilled in the art that the present invention may be practiced without the specific details presented herein. Furthermore, well known features may be omitted or simplified in order not to obscure the present invention.

A method for using dimensional and positional information as a reference to define fetal head station and position is described in PCT published patent application WO2005015499 to Paltieli et al, the disclosure of which is incorporated herein by reference. Briefly, this method includes:

1. Attaching a reference sensor to a known point of the pelvis.

2. Marking the position of three or more other known points of the pelvis by touching them with a position sensor. These position sensors provide position data in at least three degrees of freedom.

3. “Stretching” of a computerized pelvic model according to the reference sensor and the marked points.

4. Attaining the spatial position and dimensions of the pelvis and especially the pelvic inlet.

5. Calculating the fetal head station and position by a calibrated ultrasonic probe or by touching the fetal head tip with a position sensor.

6. The “stretched” pelvis model enables calculating other critical dimensions, such as the midpelvic plane and the pelvic outlet. The pelvic spatial position is constantly updated according to the attached reference sensor position.

In step 2, the positions of predetermined, known points of the female pelvis are marked by means of the position sensor. Alternatively, these points may be identified and marked by (calibrated) ultrasonic imaging, such as imaging of the symphysis pubis.

The pelvic plane may be determined by three points (e.g., touching two points and placing a reference sensor on a third point). As will be described below, in the present invention, a statistical determination of the pelvic inlet (or plane or other feature) can be made by touching just two points while the mother remains still.

It is noted that throughout the specification and claims, the terms “attaching” and “touching” will be used interchangeably. Thus, for example, attaching more than one sensor and touching with just one sensor is the same as touching with more than one sensor and attaching just one sensor.

It should be noted that when a pregnant woman comes to be checked by an obstetrician, the obstetrician does not know where her pelvic bones are from simple external observation. Thus in all of the above prior art, in order to ensure accuracy, the pelvis must first be located and mapped (that is, the spatial position of the pelvis must be first determined) each time the obstetrician examines the pregnant woman and wishes to calculate fetal head station and position. The present invention provides methods that enable the obstetrician to calculate fetal head station and position in terms of the pelvis without having to go through all the above prior art procedures, thereby providing significant time savings and increasing the comfort of the patient during examination. The methods of the present invention involve re-locating the pelvis, as is now described.

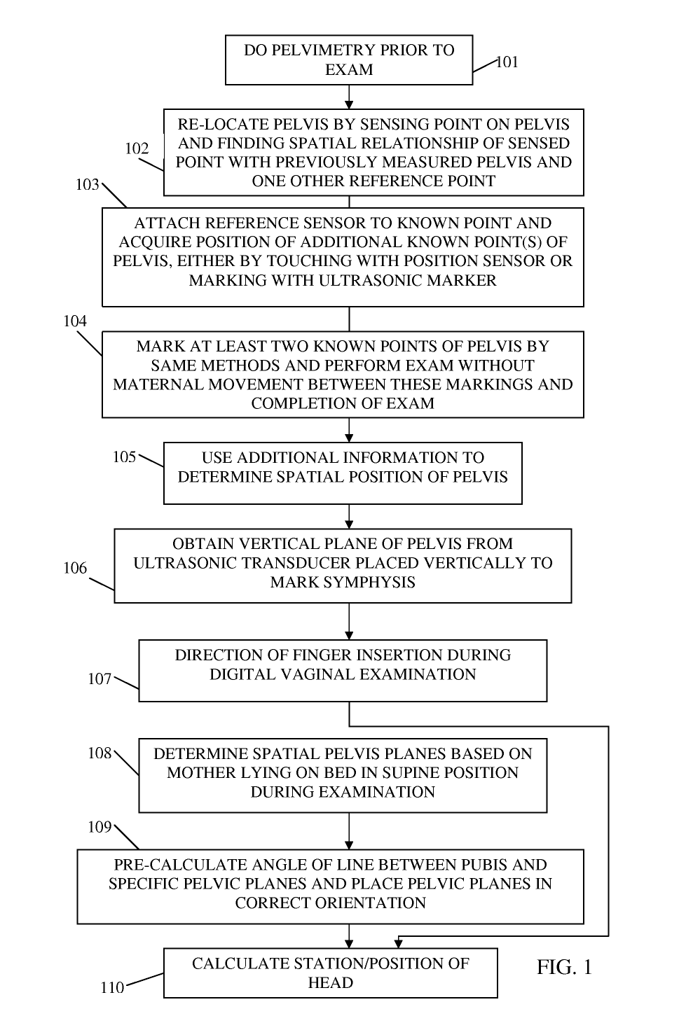

Reference is now made to FIG. 1, which illustrates a method for calculating fetal head station and position, in accordance with an embodiment of the present invention. Measurement of the pelvis (pelvimetry) may be performed at some time prior to the examination, possibly even before pregnancy (step 101). This may be done according to the steps above (without attaching a reference sensor, and by using either a (calibrated) ultrasonic probe or position sensor) or by using measurements acquired from other imaging modalities such as MRI (magnetic resonance imaging), CT (computed tomography), X-Ray or ultrasound.

Afterwards, each time the pregnant woman comes for examination, the pelvis may be re-located by sensing a point on the pelvis and finding the spatial relationship (orientation) of the sensed point with the previously measured (mapped) pelvis and one other reference point (step 102). Once this spatial relationship is known, the obstetrician now knows the present, updated orientation of the pelvis, which can be used to calculate fetal head station and position (and other parameters, such as but not limited to, cervical dilatation) as before (e.g., as described in U.S. Pat. No. 6,669,653) (step 10, below).

One way of re-locating the pelvis is by attaching a reference sensor to a known point on the patient and acquiring the position of at least one additional known point of the pelvis, either by touching that point with a position sensor or by marking the point with a (calibrated) ultrasonic marker (step 103).

Another way of re-locating the pelvis is by marking at least two known points of the pelvis by the same methods (position sensor or calibrated-ultrasonic marker) and performing the exam without maternal movement between these markings and the completion of the exam (step 104). An example would be marking two points of the symphysis pubis and the fetal head tip in the same frozen ultrasonic image (using calibrated ultrasound).

In an alternative method, only the spatial position of the pelvic inlet may be previously calculated without measuring pelvic dimensions. Two or more pelvic points may be touched with a position sensor or marked by (calibrated) ultrasound and used to generate the spatial location of specific pelvic planes (e.g., pelvic inlet, mid-pelvis).

In general, if only two points are marked on the pelvis, then additional information has to be used to determine the spatial orientation of the pelvis (step 105). This additional information may be acquired, for example, without limitation, by obtaining the vertical plane of the pelvis from a (calibrated) ultrasonic transducer placed vertically to mark the symphysis (step 106), or by the direction of finger insertion during the digital vaginal examination (step 107). Alternatively, if only two points are marked on the pelvis, the pelvic features (e.g., spatial pelvic planes) may be determined based on the fact that the mother is lying on the bed in the supine position during the examination (step 108). The statistical average angle of the line between the pubis and specific pelvic planes (e.g., pelvic inlet) may be known or pre-calculated, and the pelvic planes can be placed in the correct orientation according to this known angle and along the line created by marking two points on the pubic bone (step 109). Once the pelvic spatial position is determined, the station/position of the head can be calculated (step 110), e.g., as described in U.S. Pat. No. 6,669,653.

In general, knowledge of the spatial position of the pelvis enables determining pelvic features. “Pelvic features” or “pelvic parameters” throughout the specification and claims encompass pelvic planes, e.g., pelvic inlet, midpelvis and pelvic outlet, as well as points of interest like the ischial spines or the birth canal (a tube that passes trough the pelvic inlet plane, then through the mid-pelvis-plane, and which ends near the pelvic-outlet plane). The imaginary line that passes through the center of this tube is the birth canal path.

The prior art to Paltieli et al. determines the fetal head station in the birth canal by means of a suprapubic ultrasonic approach, including marking the fetal skull tip and the head position in the birth canal by image processing.

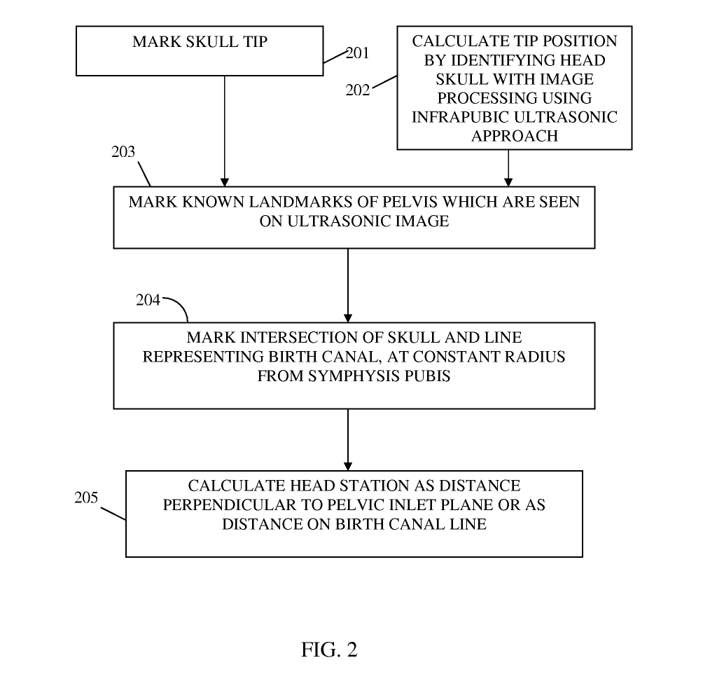

Reference is now made to FIG. 2, which illustrates a method for determining the fetal head station in the birth canal using an infrapubic ultrasonic approach, in accordance with an embodiment of the present invention. The method may include marking the skull tip (step 201) or automatically calculating the tip position by identifying the head skull with image processing using a trans-abdominal or infrapubic (trans-perineal or trans-labial) calibrated-ultrasonic approaches (step 202) (as in PCT/IL2005/000183—WO 2005/077261, the disclosure of which is incorporated herein by reference). In the infrapubic approach, the ultrasonic transducer is placed below the pubis in a median sagittal orientation and the pubic symphysis and fetal skull outline can be easily demonstrated and seen in the ultrasonic image.

Known landmarks of the pelvis which are seen on the ultrasonic image (e.g., the top and bottom edges of the symphysis pubis, and the interpubic fibrocartilaginous lamina in the center of the symphysis pubis) may be marked (step 203). Then the intersection of the skull and a line representing the birth canal, at a constant radius from the symphysis pubis, may be marked (step 204). The head station can then be calculated as a distance perpendicular to the pelvic inlet plane, to the mid-pelvis plane or as a distance on the birth canal line (step 205). This constitutes an addition to the existing method of marking the lowermost skull edge with the ultrasound placed above or below the pubic bone (PCT/IL2005/000183).

The known landmarks of the pelvis and the fetal skull may be automatically identified using common image processing techniques (as described in previous patents, such as U.S. Pat. No. 6,669,653).

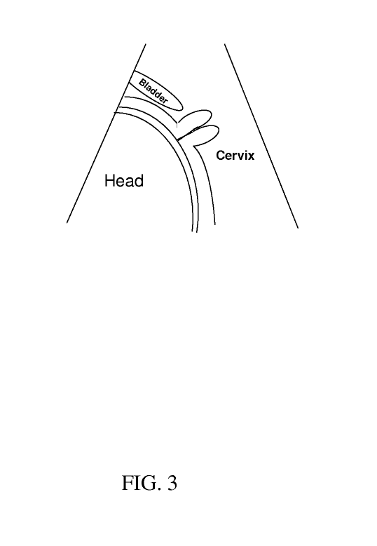

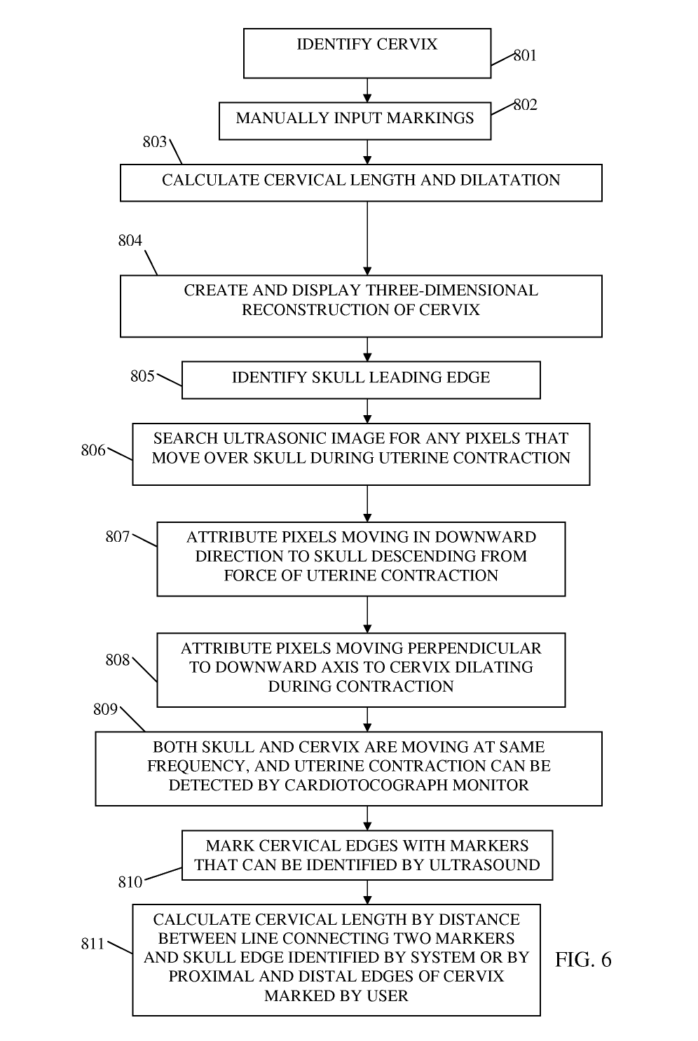

Reference is now made to FIGS. 3-5, which illustrate different stages of the cervix during labor. These figures are presented to help understand a method for ultrasonic identification of the cervix, in accordance with an embodiment of the present invention, described below with reference to FIG. 6. The method employs imaging modalities (and/or position sensors) to quantify cervical dilatation and length during labor. For example, the method may measure the dilatation and/or length of the cervix during labor utilizing a calibrated ultrasonic probe.

Measurement of the cervical length utilizing transvaginal ultrasound has been previously described in the literature and is currently the gold standard in the prediction of preterm delivery. Several publications have described the use of transperineal or translabial ultrasound for this same purpose (Kurtzmann et al 1998, Cicero et al 2001). While this technique requires more effort, it has been found to be in good correlation with transvaginal ultrasound and one can expect an 80%-95% percent success rate in demonstrating the cervix utilizing this technique. Furthermore, this technique is less invasive and more comfortable for the patient, and has less potential for causing infections.

In the early stages of labor, the cervix is an elongated cylindrical structure found below the fetal presenting part (generally the skull), as seen in FIG. 3. In the present invention, the cervix can be demonstrated by either trans-abdominal, transvaginal or infrapubic ultrasound.

The skull may be automatically identified using common image processing techniques (described in previous patents, such as U.S. Pat. No. 6,669,653). Characteristic features of the cervix may then be automatically acquired in the area below the skull edge. The cervix appears in the ultrasonic image as two echogenic layers separated by a white line.

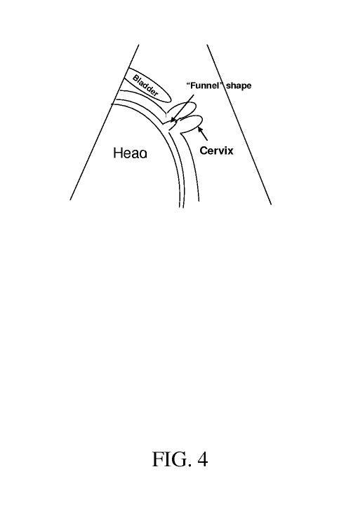

In other circumstances, the cervix can be open on its proximal end (close to the skull), as seen in FIG. 4 (“funnel” shape). In this case this hypoechogenic structure may be identified and measured to obtain a dilatation value.

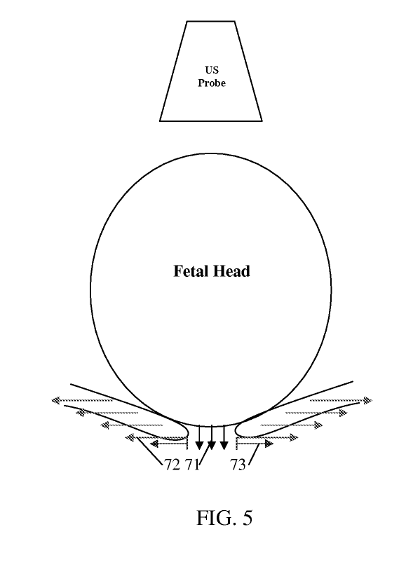

In the active phases of labor, the cervix is effaced and is a flat structure covering the skull with a round opening at the center (as seen in FIG. 5).

Reference is now made to FIG. 6, which illustrates a method for ultrasonic identification of the cervix, in accordance with an embodiment of the present invention. The cervix may first be identified (step 801), and its features may be delineated in color on the screen. The ultrasonic operator may also manually input markings, such as markings of the cervical boundaries (step 802). Upon identification of the cervix and these markings, one may calculate the cervical length and dilatation (step 803) and also create and display a three-dimensional reconstruction of the cervix (step 804).

In accordance with the method, known image processing techniques may be employed to automatically identify the skull leading edge (step 805). Then the ultrasonic image may be searched for any pixels that move over the skull during a uterine contraction (step 806). This may be accomplished, for example, by using an image processor that analyzes successive ultrasonic images and evaluates moving points in the image. Movements of the patient may be cancelled out utilizing the reference sensor and movements of the ultrasonic probe cancelled out by the sensor attached to the transducer. Pixels moving in a downward direction may be attributed to the skull descending from the force of the uterine contraction (arrows marked 71 in FIG. 5) (step 807). Pixels moving perpendicular to this downward axis may be attributed to the cervix dilating during the contraction (arrows marked 72 in FIG. 5) (step 808). Both the skull and the cervix are moving at the same frequency, and the occurrence of a uterine contraction can also be detected by a commonly used CTG (cardiotocograph) monitor (step 809).

Thus the cervix edges may be identified and the dilatation (arrows marked 73 in FIG. 5) may be measured. Since the cervix is effaced the cervix length is irrelevant.

This method can also be used in the early stage of labor. In this case cervix length need not be determined (although relevant).

In another embodiment of the present invention, the cervical edges may be marked with markers that can be identified by ultrasound (step 810). This function could be utilized both in early labor and in the active phase. The possibilities include plastic or chemical elements (e.g., a type of biological glue), a beacon transmitting sound waves, an oscillator (which can be identified by Doppler) or a capsule containing echo-lucent or echo-opaque elements (e.g., fluid, air). All of these could readily be identified by the ultrasonic transducer. These markers could be attached to the cervix using a hook or clip adapter or biological glue as described in previous patents, such as U.S. Pat. No. 6,669,653.

The dilatation may then be automatically calculated by known image processing techniques as the distance between the two markers or marked by the user. Cervical length could be calculated by the distance between the line connecting the two markers and the skull edge identified automatically by the system or by marking the proximal and distal edges of the cervix by the user (step 811).

The scope of the present invention includes both combinations and subcombinations of the features described hereinabove as well as modifications and variations thereof which would occur to a person of skill in the art upon reading the foregoing description and which are not in the prior art.

1. A method for calculating fetal parameters with reference to a female pelvis, the method comprising:
previously obtaining measurement of a pelvis of a woman;
re-locating the pelvis by sensing a point on the pelvis and finding a spatial orientation of said point with the previously measured pelvis and one other reference point; and
calculating a fetal parameter of a fetus in the woman with respect to the pelvis.

2. The method according to claim 1, wherein the fetal parameter comprises at least one of a station and a position of a head of the fetus.

3. The method according to claim 1, wherein re-locating the pelvis comprises attaching a reference sensor to a known point on the pelvis, and the other reference point comprises at least one additional known point of the pelvis.

4. The method according to claim 3, wherein the at least one additional known point of the pelvis is acquired by touching that point with a position sensor.

5. The method according to claim 3, wherein of the at least one additional known point of the pelvis is acquired by marking the point with an ultrasonic marker.

6. The method according to claim 1, further comprising using additional positional information about said pelvis to determine a spatial position of the pelvis and to calculate the fetal parameter.

7. The method according to claim 6, wherein the additional positional information is acquired by obtaining a vertical plane of the pelvis.

8. The method according to claim 6, wherein the additional positional information is acquired by a direction of finger insertion during a digital vaginal examination.

9. The method according to claim 1, wherein re-locating the pelvis comprises marking at least two known points of the pelvis, and the method comprises performing an examination of the fetus in the woman without maternal movement between the marking and completion of the examination.

10. The method according to claim 9, further comprising making a statistical average angle of a line between two pelvic features and orienting pelvic planes along said angle and a line created by marking two points on the pubic bone.

11. The method according to claim 1, wherein calculating the fetal parameter of the fetus in the woman comprises:
making an ultrasonic image that includes the birth canal and the pelvis of the woman;
identifying a tip of a skull of the fetus;
marking known landmarks of the pelvis associated with the birth canal, which are seen on the ultrasonic image; and
calculating head station by correlating the known landmarks with the tip of the skull.

12. The method according to claim 11, wherein correlating the known landmarks with the tip of the skull comprises marking an intersection of the skull and a line representing the birth canal.

13. The method according to claim 11, wherein the head station is calculated as a distance to a pelvic feature.

14. The method according to claim 11, wherein the head station is calculated as a distance on a line of the birth canal.

15. The method according to claim 11, further comprising processing said ultrasonic image to automatically identify known landmarks of the pelvis and the skull.

16. A method for determining cervical information associated with a woman pregnant with a fetus, the method comprising:
identifying a cervix on an ultrasonic image;
marking boundaries of the cervix;
calculating cervical length and dilatation based on the identified cervix and its boundaries; and
creating and displaying a three-dimensional reconstruction of the cervix.

17. The method according to claim 16, further comprising identifying a leading edge of a skull of the fetus.

18. The method according to claim 16, further comprising searching the ultrasonic image for a first set of pixels which move over a skull of the fetus during a uterine contraction.

19. The method according to claim 18, comprising attributing the first set of pixels as relating to the skull descending from a force of the uterine contraction.

20. The method according to claim 18, comprising attributing a second set of pixels in said ultrasonic image, which move generally perpendicular to the first set of pixels, as related to the cervix dilating during the uterine contraction.

21. Apparatus for determining dimensions and spatial position of a female pelvis, the apparatus comprising:
a reference sensor attachable to a known point on a female pelvis of a pregnant woman;
a position sensor for acquiring a position of at least one additional known point of the pelvis; and
ultrasonic markers of at least two known points of the pelvis.

22. Apparatus for determining fetal head station in a birth canal associated with a female pelvis of a woman pregnant with a fetus, the apparatus comprising:
an ultrasonic image that includes the birth canal and the pelvis of the woman;
an ultrasonic identifier of a tip of a skull of the fetus;
ultrasonic markers of known landmarks of the pelvis associated with the birth canal, which are seen on the ultrasonic image; and
a processor for calculating head station that correlates the known landmarks with the tip of the skull.

23. The apparatus according to claim 22, wherein said processor is adapted to process said ultrasonic image to automatically identify known landmarks of the pelvis and the skull.

24. Apparatus for determining cervical information associated with a woman pregnant with a fetus, the apparatus comprising:
an ultrasonic image that includes a cervix identified thereon;
a processor for calculating cervical length and dilatation based on the identified cervix and its boundaries, said processor being operative to create a three-dimensional reconstruction of the cervix.

25. The apparatus according to claim 24, further comprising a display for displaying the three-dimensional reconstruction of the cervix.
